(19) United States
(12) Patent Application Publication
(10) Pub. No.: US 2014/0010712 A1
(43) Pub. Date: Jan. 9, 2014

(54) APPARATUS FOR DETECTING A SELECTED LIQUID CONTAMINANT

(71) Applicant: Petro Barrier Systems Inc., Victoria (CA)

(72) Inventors: Imtehaze HEERAH, Victoria (CA); Iain MUIR, Saanich (CA); William J. SPAULDING, Victoria (CA)

(21) Appl. No.: 13/935,598

(22) Filed: Jul. 5, 2013

(30) Foreign Application Priority Data
Jul. 5, 2012 (CA) 2782675

(51) Int. Cl. G01N 33/18

(52) U.S. Cl. CPC G01N 33/1826
USPC 422/69

(57) ABSTRACT

An apparatus for detecting a selected liquid contaminant includes a material holder and a reactive material held by the material holder. The reactive material absorbs a selected liquid contaminant with a measurable change reaction occurring as a result of such absorption. A signal generator is provided for generating a signal upon the measurable change reaction exceeding a preset threshold.

### FIELD

There is described an apparatus for detecting a selected liquid contaminant. The apparatus was developed for the purpose of detecting the presence of liquid hydrocarbons in water, but can be adapted to detect other liquid contaminants.

### BACKGROUND

U.S. Pat. No. 7,014,755 (Muir et al), entitled “Filtration and plug drain device for containing oil and chemical spills”, describes a filtration apparatus with a filtration material that has particular properties. The filtration material is hydrophobic; allowing water to pass freely through in the absence of oil or chemical contaminants. The filtration material absorbs oil or chemical contaminants. This results in small quantities of oil and chemical contaminants being absorbed while water continues to pass through. However, the flow of all liquids is blocked entirely when oil or chemical contaminants are present in large quantities. The Muir apparatus is installed in municipal drains in order to prevent oil or chemical contaminants from entering. The apparatus is installed at sites, such as service stations and parking lots, where oil spills occasionally occur. At the present time, each drain installation must be periodically visited and the filtration apparatus checked to determine the state of the filtration material. What is required is a method and apparatus for remotely monitoring the condition of the filtration material.

### SUMMARY

Remote monitoring of filter elements has previously been described in patents, such as U.S. Pat. No. 6,377,171 (Fewel). Sending signals to remote monitoring stations is well known in a number of industry sectors. The problem was establishing a parameter that could be monitored and would provide a reliable indication as to the state of the filter material. A research project was undertaken to compare samples of new filter material, with samples of filter material that had been exposed to oil or chemical contaminants. The properties studied included dielectric properties, optical transparency and volumetric expansion. It was determined that there was a change in all of those properties, with volumetric expansion being preferred as the most readily monitored. This lead to further research to develop a workable apparatus for detecting liquid contaminants which could be incorporated into the filtration apparatus. Although the apparatus was developed for use in detecting the presence of hydrocarbons, it was determined that the same principles could be used in detecting the presence of other liquid contaminants.

According to the present invention there is an apparatus for detecting a selected liquid contaminant. The apparatus includes a material holder and a reactive material held by the material holder. The reactive material absorbs a selected liquid contaminant with a measurable change reaction occurring as a result of such absorption. A signal generator is provided for generating a signal upon the measurable change reaction exceeding a preset threshold.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a side elevation view, in section, of an analog apparatus for detecting a liquid contaminant based upon volumetric expansion of a reactive material.

FIG. 2 is a top plan view of an indicator element from the analog apparatus illustrated in FIG. 1.

FIG. 3 is a side elevation view, in section of a binary apparatus for detecting a liquid contaminant based upon volumetric expansion of a reactive material.

FIG. 4 is a top plan view of an indicator element from the binary apparatus illustrated in FIG. 3.

FIG. 5 is a side elevation view, in section, of an apparatus for detecting a liquid contaminant based upon changes in optical transparency of a reactive material.

FIG. 6 is a side elevation view, in section, of an apparatus for detecting a liquid contaminant based upon changes in capacitance of a reactive material.

FIG. 7 is a top plan view, of a filtration apparatus utilizing one of the apparatus for detecting a liquid contaminant illustrated in FIG. 1 through FIG. 6.

FIG. 8 is a side elevation view, in section, of an in ground tank monitoring application utilizing one of the apparatus for detecting a liquid contaminant illustrated in FIG. 1 through FIG. 6.

FIG. 9 is a side elevation view, in section, of a ship bilge water monitoring application utilizing one of the apparatus for detecting a liquid contaminant illustrated in FIG. 1 through FIG. 6.

FIG. 10 is a side elevation view, in section, of a pipe leakage monitoring application utilizing one of the apparatus for detecting a liquid contaminant illustrated in FIG. 1 through FIG. 6.

### DETAILED DESCRIPTION

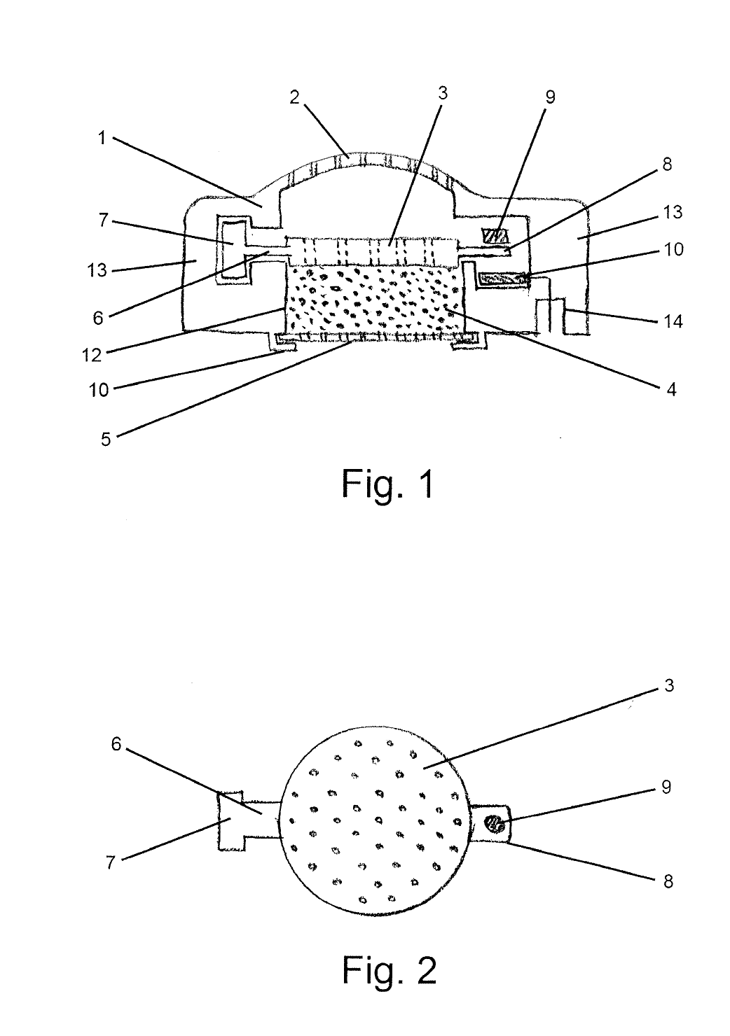

A series of variants on an apparatus for detecting a selected liquid contaminant and potential applications for such apparatus will now be described with reference to FIG. 1 through 10.

### Liquid Hydrocarbon Reactive Material (LQHRM)

Co-polymers of styrene and olefins such as ethylene, propylene, butylene, isobutylene, butadiene and elastomers including ethylene/propylene, ethylene/butylenes and isoprene have shown a propensity to react with hydrocarbon oils. These hydrocarbons include motor, transformer and hydraulic oils, gasoline, diesel and solvents such as xylene, toluene, mineral spirits, turpentine and varsol. These co-polymers are produced as powders or granular pellets; when hydrocarbon oils come in contact with these resins they are absorbed into the solid granules to produce a solid gel which can quickly swell in volume and become a rubber like solid. These styrenic co-polymers vary in their reactivity with hydrocarbon oils and the extent of the swelling occurring. Studies have shown that the resins which exhibit this “swelling” effect are predominantly co-polymers of styrene, ethylene/propylene and styrene-ethylene butylene of different compositions varying in the concentrations of the polyolefin elastomers.

In North America, these co-polymers are marketed as the G-series of Kratons. While many Kratons such as G1650, G1651, G1652 G1654, G1701, G1702, G1726 are effective in reacting with hydrocarbon oils causing an expansion in volume, G1654 was shown to be significantly better than the others, expanding by 100% or more of the original volume and absorbing as much as 10 times its weight of hydrocarbon oils. G1654 is a linear tri-block co-polymer based on 29-33% styrene and ethylene/butylenes (S-EB-S). Its absorptive and “swelling” features are 60-80% greater than other G series Kratons evaluated and the change of state from a granular powder to a solid gel occurs within less than 30 seconds of contact with hydrocarbon oils.

When allowed to absorb a quantity of liquid hydrocarbon it goes through a number of physical changes that can be used to detect the presents of liquid hydrocarbon. In general terms, when LQHRM absorbs a quantity of liquid hydrocarbon it changes from a course powder to a continuous gel. The material is hygroscopic and in powder form will allow liquids such as water to pass through relatively unimpeded but the LQHRM will not absorb any of the water itself. When LQHRM comes in contact with liquid hydrocarbon it readily absorbs it and exhibits several physical, electrical and optical changes that can be used to infer the presents of liquid hydrocarbon.

The selection of suitable LQHRM is described in the Applicant's U.S. Pat. No. 7,014,755. The relevant portions of which are reproduced here for sake of completeness:

To test whether a candidate material is suitable for use as filter material, filter/plug material or plugging material for a target hydrocarbon, a small sample of the candidate material was placed in a container and the target hydrocarbon was added until the candidate material is essentially saturated, that is, it ceased to rapidly take up the target hydrocarbon. For materials that were ultimately considered to be suitable for use with a particular target hydrocarbon, this typically occurred within several seconds. The state of the candidate material/target contaminant combination was evaluated at 1 minute, 10 minute and 24 hour intervals through visual observation and physical manipulation.

When testing to determine whether selected styrenic block copolymers were suitable for use as filter material, filter/plug material or plugging material for a target hydrocarbon, results indicating that the material would be suitable included the following: a) for a plugging material: i) at 1 minute, an essentially impermeable plug would have formed, which could be hard or solid, rubbery or spongy, or similar to sticky cooked rice; ii) at 10 minutes, the plug would be stable or would have become more solid; and iii) at 24 hours, the plug would be stable or would have become more solid; b) for a filter/plug material: i) at 1 minute, the material is permeable; the material has absorbed all of the added target contaminant and may be able to absorb more over time; the surfaces of the constituent particles of the material may be sticky and there may be some clumping of the particles; ii) at 10 minutes, the material is still permeable and preferably still absorbent, though the constituent particles may be more sticky and there may be more clumping; and iii) at 24 hours, the material is still permeable and preferably still absorbent; the constituent particles may be more sticky and there may be more clumping, but they have not formed a solid; and c) for a filter material: i) at 1 minute, the material is permeable, with minimal change to the surfaces of the constituent particles (i.e. a minimal increase in “stickiness”); ii) at 10 minutes, the material is essentially stable, in that it is permeable and the surfaces of the constituent particles are essentially unchanged; and iii) at 24 hours, the material is essentially stable, in that it is permeable and the surfaces of the constituent particles are essentially unchanged.

In some cases, different mixtures of two or more styrenic block copolymers may provide the desired characteristics for each of the filter material, filter/plug material and plugging material. For example, a candidate material for a plugging material that reacts when exposed to the target hydrocarbon by rapidly forming a solid, but permeable (due to internal voids), agglomeration may be combined with another material that initially reacts to the target hydrocarbon by forming a syrupy plug, in that a mixture of these materials would form a suitable plug, with the first material providing a solid framework for the plug and the second material filling and sealing the voids within the framework.

In an exemplary embodiment of the filter/plug drain insert 20 targeted at the oil used in electrical transformers (commonly referred to as transformer oil): a) the filter pellets 54 consist of a 50:50 mixture of: i) a pelletized two-component mixture comprising: A) 60% a styrene-ethylene/butylene-styrene copolymer (conventionally referred to by the acronym SEBS) comprising 30% a linear, 0% diblock, copolymer of styrene and 70% ethylene/butylene rubber (Kraton™ G1650 or equivalent); and B) 40% polypropylene; and ii) a pelletized SEBS comprising 30% a linear, 70% diblock, copolymer of styrene and 70% ethylene/butylene rubber (Kraton™ G1726 or equivalent); b) the filter/plug grains 56 consist of a granular three-component mixture comprising: i) 45% a styrene-ethylene/propylene copolymer (conventionally referred to by the acronym SEP) comprising 37% a 100% diblock copolymer of styrene and 63% ethylene/propylene rubber (Kraton™ G1701 or equivalent); ii) 20% a SEBS comprising 30% a linear, 0% diblock, copolymer of styrene and 70% ethylene/butylene rubber (Kraton™ G1650 or equivalent); and iii) 35% a SEBS comprising 31% a linear, 0% diblock, copolymer of styrene and 69% ethylene/butylene rubber (Kraton™ G1654 or equivalent); and c) the rapid plugging material 58 consists of a two-component mixture of granular copolymers comprising: i) 50% a SEP comprising 37% a 100% diblock copolymer of styrene and 63% ethylene/propylene rubber (Kraton™ G1701 or equivalent); and ii) 50% a SEBS comprising 30% a linear, 0% diblock, copolymer of styrene and 70% ethylene/butylene rubber (Kraton™ G1650 or equivalent).

Each of the individual materials for use with transformer oil, was tested using the testing procedure outlined above. The materials (identified below primarily by their Kraton™ product numbers for convenience) were observed to perform as follows: a) Kraton™ G1650: i) at 1 minute—all oil is easily absorbed; has the appearance of wet sugar; slightly sticky to the touch; ii) at 10 minutes—material is a somewhat loose sticky mass; not a solid plug; and iii) at 24 hours—fairly good rubbery plug formed made of relatively loose grains that can easily be pulled apart though; b) Kraton™ G1654: i) at 1 minute—all oil has been easily absorbed; has the appearance of wet sugar; slightly sticky to the touch; ii) at 10 minutes—no change; and iii) at 24 hours—no change; all oil remains contained, but material is loose, not a solid plug; c) Kraton™ G1701: i) at 1 minute—all oil absorbed-material has turned “soupy” and “syrupy”; not a solid plug; ii) at 10 minutes—similar to material at 1 minute; somewhat gummy; iii) at 24 hours—wet and syrupy plug; and d) Kraton™ G1726: i) at 1 minute—no plugging; appears that some slight swelling of pellets may be occurring; ii) at 10 minutes—pellets swollen and starting to stick together; and iii) at 24 hours—good hard solid plug and d) the pelletized mixture comprising 60% Kraton™ G1650 and 40% polypropylene: i) at 1 minute—pellets suspended in oil; no change to pellets; ii) at 10 minutes—slight swelling of pellets; and ii) at 24 hours—pellets swollen and sticking together, but can be easily pulled apart from each other.

Similar testing of candidate materials was used in the selection of materials targeted at conventional vehicle fuels (diesel and gasoline): a) the filter pellets 54 consist of a pelletized SEBS comprising 30% a linear, 70% diblock, copolymer of styrene and 70% ethylene/butylene rubber (Kraton™ G1726 or equivalent); b) the filter/plug grains 56 is a granular two-component mixture comprising: i) 3 parts a SEBS comprising 31% a linear, 0% diblock, copolymer of styrene and 69% ethylene/butylene rubber (Kraton™ G1654 or equivalent); and ii) 1 part a SEP comprising 37% a 100% diblock copolymer of styrene and 63% ethylene/propylene rubber (Kraton™ G1701 or equivalent); and c) the rapid plugging material 58 consists of a three-component mixture comprising: i) 1 part a pelletized SEBS comprising 30% a linear, 70% diblock, copolymer of styrene and 70% ethylene/butylene rubber (Kraton™ G1726 or equivalent); ii) 2 parts a granular styrene-butadiene-styrene copolymer (conventionally referred to by the acronym SBS) comprising 31% a linear, 16% diblock, copolymer of styrene and 69% ethylene/butadiene (Kraton™ D1101 or equivalent); and iii) 2 parts a SEBS comprising 33% a linear, 0% diblock, copolymer of styrene and 67% ethylene/butylene rubber (Kraton™ G1651 or equivalent).

The materials of each of the above-described transformer-oil-targeted and vehicle-fuels-targeted embodiments are of the sort that as well as filtering the target contaminant also have a slow plugging reaction (as described generally above). As the materials of these embodiments absorb contaminants, they gradually swell and as they continue to absorb more contaminants they may eventually swell sufficiently to fill the interstitial spaces, thus impeding further flow of liquid through the material.

The physical changes exhibited by LQHRM upon exposure to liquid hydrocarbon include but are not limited to:
Gelification: the process of turning from a powder to the continuous gel
Volumetric Expansion: LQHRM demonstrates a substantial expansion
Optical change from opaque to translucent
Changes in its dielectric properties

While any one or more of these property changes can be used to detect the presents of liquid hydrocarbon the two detailed instances, namely “Analog Variant” and “Binary Variant”, described in this patent focus on the combined effect of Gelification and Volumetric Expansion and their use in drain protection. Other instances of variants of the sensor, that take advantage of Optical and dielectric property changes, are described in “Other Sensor Variants”.

Several additional instances of sensor usage are provided in “Other Liquid Hydrocarbon Sensor Applications”. These variants and the drain protection variants could use any one or combination of the sensor variants described.

### Analog Variant

The purpose of the instance shown in FIG. 1 and FIG. 2, is to produce an electrical voltage output that varies in proportion to the volume of liquid hydrocarbon that has passed in the sensor. FIG. 1 shows a sectional view of the analog variant and functions as follows.

Non-hydrocarbon liquids are allowed to pass through sensor by entering the Sensor Case (1) through the matrix of small holes formed in the Inlet Screen (2), flowing or dripping through the small holes formed in the Perforated Pressure Disk (3), moving through the LQHRM layer (4) and finally leaving the sensor through the small openings in the

Discharge Screen (5). As the LQHRM layer (4) is not activated by anything other than liquid hydrocarbons none of the sensing elements housed within the Sensor Case (1) are triggered or activated during the transmission of Non-hydrocarbon liquids through the sensor.

Liquid hydrocarbons are allowed to enter the Sensor Case (1) through the matrix of small holes formed in the Inlet Screen (2). They then flow or drip through the small holes formed in the Perforated Pressure Disk (3). As the liquid hydrocarbon moves into the LQHRM layer (4) the LQHRM (4) reacts to the presents of liquid hydrocarbon in two ways: It turns from a fine powder to a gel; and, its volume markedly increases in proportion to the volume of oil it absorbs. As the LQHRM (4) expands it presses up onto the underside of the Perforated Pressure Disk (3), which functions as an indicator element. The Perforated Pressure Disk (3) is supported at one end by a Cantilever Spring Element (6) that is in turn firmly attached to the Sensor Case (1) by the Spring Element Rigid Attachment (7). The Magnetic Support Extension (8) protrudes from the free end of the Perforated Pressure Disk (3). Attached to the Magnetic Support Extension (8) is a Magnet (9). Located directly below the Magnetic Support Extension (8) and the Magnet (9) is a Hall Effect Sensor (10). The Hall Effect Sensor (10) is fixed to the inside surface of the Sensor Case (1). As the expanded LQHRM (4) presses up on the underside of the Perforated Pressure Disk (3) the Perforated Pressure Disk (3) is rotated upward against the resistive toque imposed on the Perforated Pressure Disk (3) by the Cantilever Spring Element (6). As the Perforated Pressure Disk (3) rotates upward the Magnet Support Extension (8) the Magnet (9) associated with it are raised moving them farther away from the Hall Effect Sensor (10). The voltage output of the Hall Effect Sensor (10) thus changes indicated the relative distance between the Hall Effect Sensor (10) and the Magnet (9). Because the change in electrical output from the Hall Effect Sensor (10) has been caused by the introduction of liquid hydrocarbon to the sensor the voltage output can be used to communicate that liquid hydrocarbon has entered the Sensor Case (1) and, with sufficient calibration, the approximate quantity of liquid hydrocarbon that has entered the Sensor Case (1).

While the sensor is in service, debris is kept out of the sensor in four ways: the matrix of holes formed into the inlet screen (1) are sufficient small to ensure that larger debris cannot enter the Sensor Case (1); the domed top of the Sensor Case (1) works to allow debris that accumulates on the outside of the Sensor Case (1) will wash away over time and thus self-clear the passageways for liquid hydrocarbon and other liquids that lead to the interior of the sensor; should some debris eventually enter the Sensor Case (1) the small holes in the Perforated Pressure Disk (3) will prevent the debris from contaminating the LQHRM (4); and, an external filter fabric (not shown) will surround the Sensor Case (1) providing a primary filter barrier against the entry of solid debris into or onto the sensor.

On the underside of the Sensor Case (1) is the Discharge Screen (5). The Discharge Screen (5) has the dual function of holding the LQHRM (4) material in place and allowing liquids other than hydrocarbons from exiting the Sensor Case (1). The Discharge Screen (5) is held in place by the Discharge Screen Track and Support (10) that has been formed on the three sides of the Discharge Screen's (5) perimeter. The Discharge Screen Track and Support (10) allow the screen to be slid out and returned in position during manufacture or maintenance. During manufacture of the sensor it is important to place a consistent volume of LQHRM (4) into the sensor. An additional function of the Discharge Screen (5) is to enable a precise volume of LQHRM to be loaded into the LQHRM Cavity (12) during manufacture. During manufacture the Discharge Screen (5) is removed and LQHRM is added to the LQHRM Cavity (12) until it is over full. The Discharge Screen (5) is slid into place cutting the LQHRM mound leaving a specific amount of LQHRM in the LQHRM Cavity (12). One additional advantage of arranging the Discharge Screen (5) in this way is that the sensor can be easily reconditioned for re-use. Once the LQHRM has been saturated with oil it becomes a semi-solid gel it must be reconditioned. Reconditioning the sensor is simply a matter of sliding the Discharge Screen (5) out, lifting the plug of now gelled LQHRM (4) out of the LQHRM Cavity (12) and refilling the LQHRM Cavity (12) with fresh LQHRM (4). The Discharge Screen (5) is slide back into place and the sensor is ready for re-use.

In many applications the Sensor will be mounted into an external mounting structure along with other Liquid Hydrocarbon Sensors. To enable easy and secure installation, Sensor Mounting Tabs (13) have been added to the ends of the Sensor Case (1). These wedge themselves into the external mounting structure. The integrated, downward facing, Electrical Connector (14) is used to electrically associate the sensor with a mating electrical connector in the external mounting structure. The combination of the Sensor Mounting Tabs (13) and the integrated Electrical Connector (14) allow each sensor to be installed and removed from the external mounting structure during manufacture or in the field, without the use tools and without the need for any final adjustments.

For this and the variations that will hereinafter be described, the sensor case merely serves as a material holder and can take a number of forms. It need not be a rigid case, as illustrated, but could be a flexible bag, as long as the components that collectively cooperate to serve as the signal generator are compatible with the material holder. For example, a Kevlar (Trademark) bag could be used with a pressure sensor that detects volumetric expansion of the reactive materials within the bag. The signal that is generated can take a number of forms. The signal can be physical, mechanical, electrical or optical. The intent is merely to draw to the attention to whomever is tasked with monitoring the apparatus, that a change reaction has occurred.

### Binary Variant

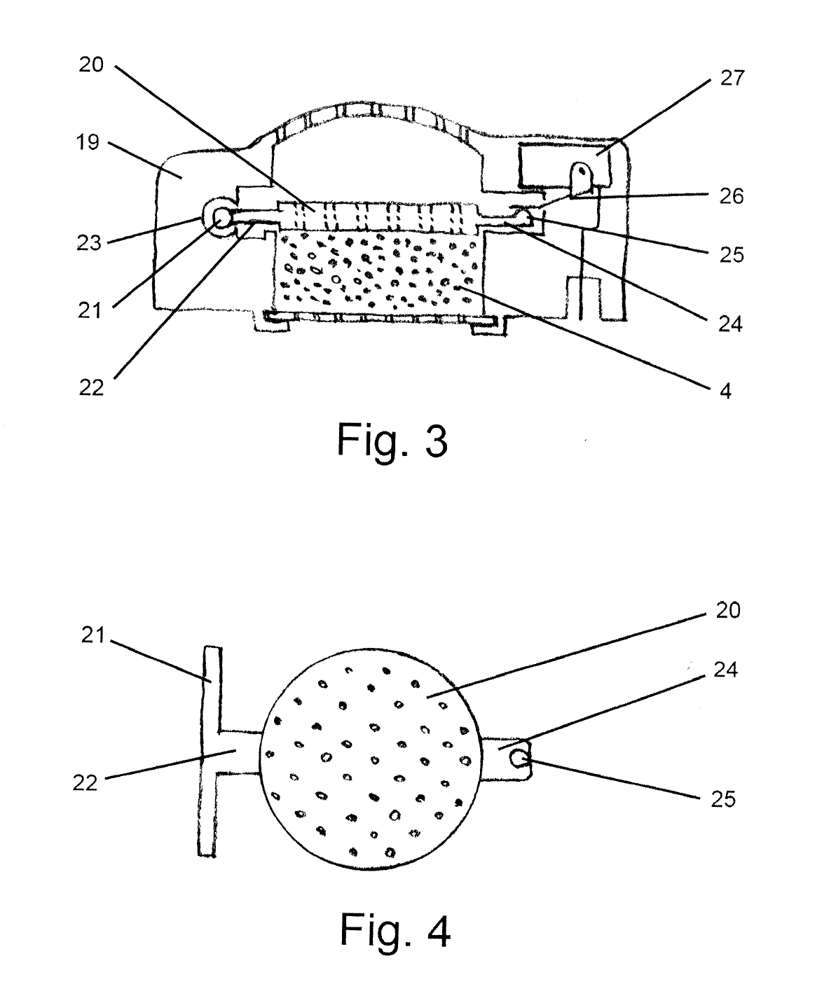

The Binary Variant of the Liquid Hydrocarbon Sensor, FIG. 3 and FIG. 4 differs from the Analog Variant (described above) in that it uses an electrical switch to communicate the present of liquid hydrocarbon. The electrical action of the Binary Variant is simply to open a switch when liquid hydrocarbon has entered the sensor and to close a switch when no liquid hydrocarbon is present. Many of the components used in the Binary Variant are the same as the Analog Variant but the primary differences involve the design and function of the Perforated Pressure Screen (20) and components associated with it. A functional description follows.

When liquid hydrocarbon enters the sensor and passes into the LQHRM (4) the resulting upward expansion of the LQHRM (4) presses against the underside of the Perforated Pressure Screen (20). In response the Perforated Pressure Screen (20) rotates upward around the Hinge Pin (21) that is attached to the Perforated Pressure Screen (20) via the Hinge Extension (22). The Hinge Pin (21) is supported and allowed to freely rotate in the Hinge Pin Support (23) that is part of the Sensor Case (19). As the Perforated Pressure Screen (20) rotates its Extension Arm (24) lifts and moves the Force Point (25) upward against the Switch Arm (26). The Switch Arm (26) then moves and, given sufficient force and motion will activate the Electrical Switch (27). The combination of Force Point (25) deflection and generated force indicate that a threshold volume of liquid hydrocarbon has entered the sensor. Once this threshold has been reached the Electrical Switch (27) will be activated. Internal to the Electrical Switch (27) is a spring that resists the motion of the Force Point (25). Resisting the motion of the Force Point (25) allows the sensor to be loaded with a repeatable quantity of LQHRM during manufacture, prevents the sensor from getting out of adjustment during transport and allows the Electrical Switch (27) to be activated when a consistent volume of liquid hydrocarbon has entered the sensor.

### Sensor Network Installation in a Drain

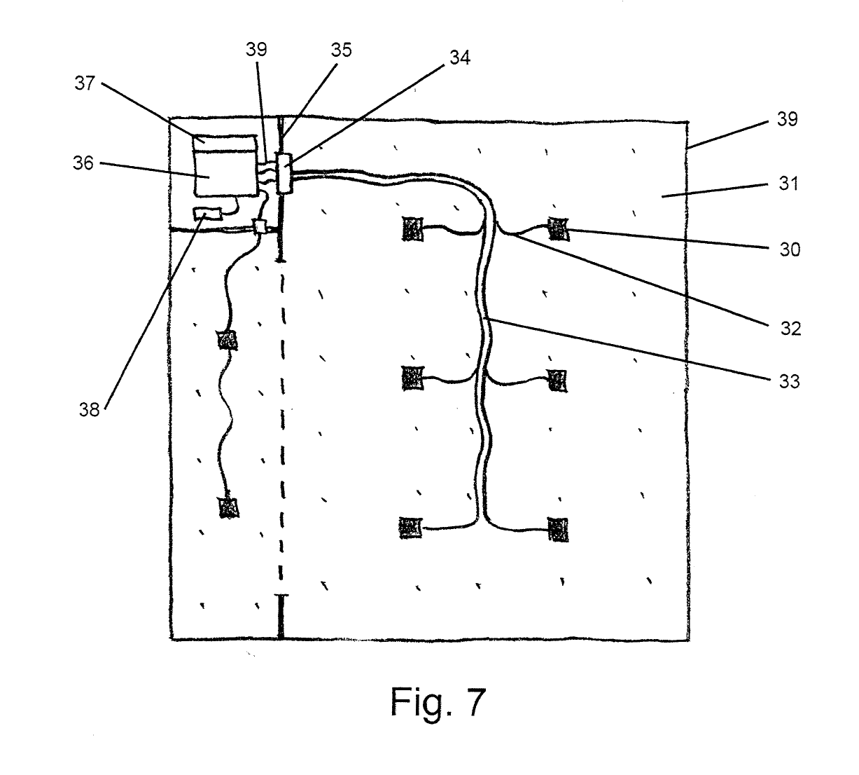

One way the liquid hydrocarbon sensors described above can be used is to detect the presence of liquid hydrocarbon in a drain. A specific instance, illustrating the sensor's use in a specially protected drain filter, is shown in FIG. 7.

Each Liquid Hydrocarbon Sensor (30) is mounted onto a Perforated Support Screen (31). The Sensor (30) is mounted firmly to the Perforated Support Screen (31) by two means: the Sensor (30) is partially inserted into a recess molded into the Perforated Support Screen (31) thus holding it in place physically; and, as the Sensor (30) is inserted into the mounting recess it also plugs into an electrical receptacle molded into the Perforated Support Screen (31). In this way each Sensor (30) is connected to the Perforated Support Screen (31) both physically and electrically. Integral to the Perforated Support Screen (31) are a network of wires. The wire path starts at the Sensor (30) and runs via Sensor Wires (32) into larger grouping of Sensor Wires, the Sensor Bus (33). The Sensor Bus (33) then runs to the edge of the Perforated Support Screen (31) and terminates at a multi-pin Waterproof Connector (34). The Waterproof Connector (34) is in two parts: one part is mounted to the Perforated Support Screen (31); and, the other part is mounted into the wall of the Electrical Housing (35) that serves to contain and protect the electronic components that make up the Communication Electronics (36), the battery (37) and the Antenna (38). During installation the both parts of the Waterproof Connector (34) are brought together to form a continuous electrical connection between the sensors (30) and the electronics contained within the drain's Waterproof Housing (35). Inside the Waterproof Housing (35) the electrical signal from the Sensors (30) runs from the Waterproof Connector (34) along an Internal Electrical Bus (39) connecting finally to the Communications Electronics (36).

The function of the Communications Electronics (36) is twofold. Firstly, the Communications Electronics (36) periodically poles the condition of each of the Sensors (30). Poling takes place one sensor at a time. The returning signal from each Sensor (30) may be an analog voltage or a binary voltage signal, depending on the variant of Sensor (30) used. In either case the returning signal indicates whether the Sensor (30) has absorbed liquid hydrocarbon. Secondly, the Communications Electronics (36) activates its built-in wireless communication system and reports the condition of each sensor (30) to the drain maintenance staff so that, in the case of the introduction of liquid hydrocarbon into the drain, they can take appropriate and immediate action. A low profile antenna (38), installed at the top of the waterproof housing (35) acts as the primary link that enables a reliable wireless communication path to an external monitoring station. Power from the battery (37) is applied to the Communication Electronics (36) only as required by the poling schedule. In this way battery life is maximized. In some installations a small solar voltaic battery charger (not shown) will be installed at the top of the Waterproof Housing (36) that will provide a sufficient slow charge to the Battery (37) and enable the Communications Electronics (36) to monitor and report the condition of the Sensors (30) and Drain (39) for an indefinite period. Within the Communications Electronics (36) a microcontroller will coordinate all activities of the system, pole and measure the output from each sensor (30) and initiate and format all wireless communications signals. A separate communications module will be included within the Communications Electronics (36). It will utilize the cellular network, wifi or some other wireless or wired communications platform best suited to the conditions found at the Drain (39) location.

### Other Sensor Variants

### Optical Sensor Variant

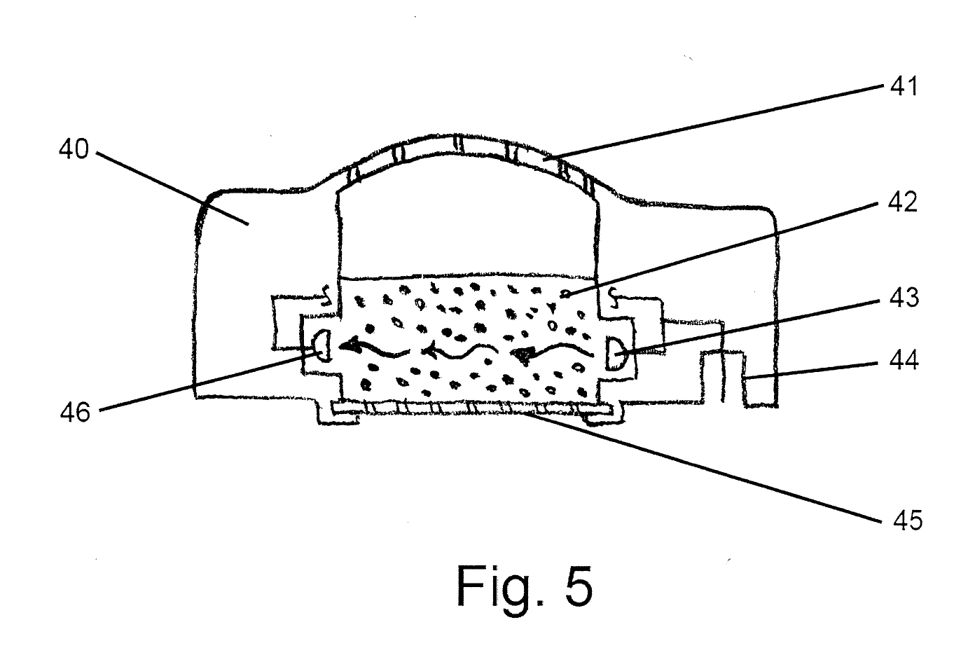

FIG. 5 shows an instance of the liquid hydrocarbon sensor that utilizes changes in the transparency of the LQHRM. When the LQHRM has not been exposed to liquid hydrocarbon it takes the form of an opaque course grained powder. In this state the LQHRM is a poor transmitter of light. When the LQHRM has been exposed to liquid hydrocarbon it changes in to a continuous translucent gel with sufficient translucents that sufficient light can pass through to alter the state of an light detector. No such optical changes take place within the LQHRM when it is exposed to other liquids such as water. By producing a sensor with a light source, a layer of LQHRM and a light detector the presents of liquid hydrocarbon can be detected.

In the instance shown in FIG. 5 liquid enters the sensor through the Inlet Screen (41) and then passes through the LQHRM (42). If the liquid is water it will pass all the way through the sensor leaving via the Discharge Screen (45). Should the liquid be liquid hydrocarbon the LQHRM (42) will absorb it and react to it in such a way that the LQHRM (42) will form a translucent continuous gel. A Light Source (43) periodically shines light through the LQHRM (42). At the same time the Light Source (43) becomes active a Light Detector (46), placed so as to be out of direct view of the Light Source (43), begins to measure the level of transmitted light. If the LQHRM (42) has not been exposed to liquid hydrocarbon the Light Detector (46) will detect no discernible transmitted light and report that no liquid hydrocarbon is present. If the LQHRM (42) has absorbed oil some light will be transmitted all the way through the LQHRM (42) and the Light Detector (46) will respond by reporting that Liquid Hydrocarbon has entered the sensor. Once the condition of the sensor has been determined electronics within the sensor will transmit that information out of the sensor via the Electrical Connector (44), through electrical interconnections between the network of sensors, to the staff or system tasked with the overall monitoring.

### Dielectric Variant

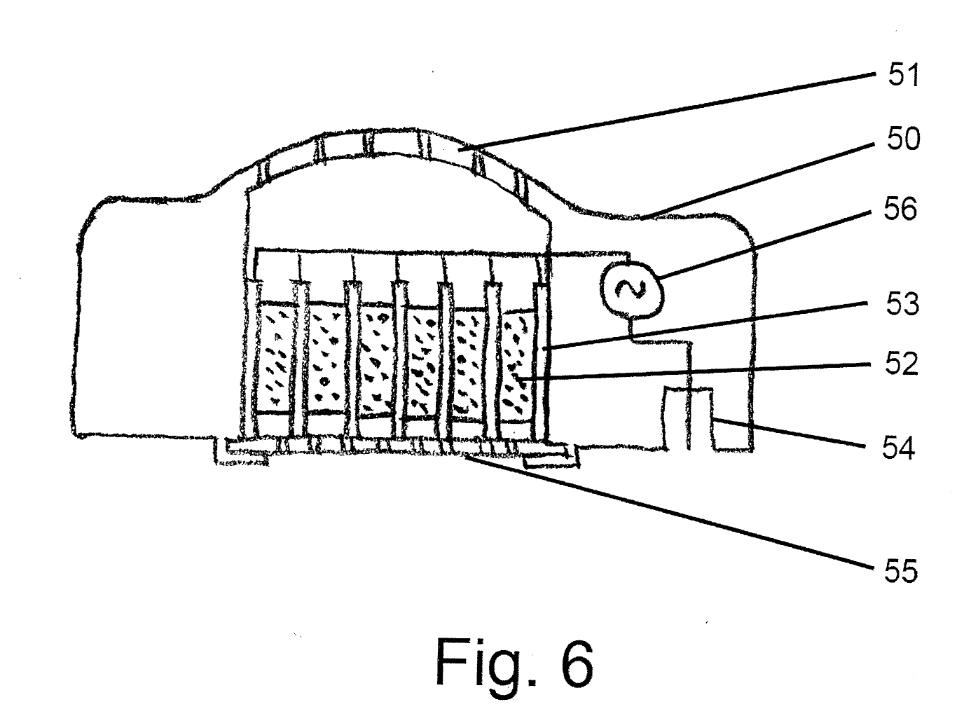

FIG. 6 shows an instance of liquid hydrocarbon sensor that utilizes changes in the dielectric properties of the LQHRM. This variant is constructed much like a standard electrolytic capacitor in that a multitude of thin layers of LQHRM (52) are separated by thin Electrically Conductive Plates (53). The Electrically Conductive Plates (53) are connected in such a way that they can be attached to capacitance detector (56). Periodically the capacitance detector (56) places a rapidly oscillating voltage onto the Electrically Conductive Plates (53) and measures the overall electrical impedance of the LQHRM (52)/Electrically Conductive Plate (53) assembly. When the LQHRM (52) is dry its dielectric constant is quite low, when the LQHRM (52) is saturated with water its dielectric constant is quite high and when the LQHRM (52) is saturated with liquid hydrocarbon that dielectric constant takes on a medium magnitude. These alterations in dielectric constant enable the capacitance detector (56) to distinguish between these three conditions.

As in the other sensor variants, liquid enters the sensor through the Inlet Screen (51), passes down through the LQHRM (52)/Electrically Conductive Plates (53) assembly. If the liquid is not liquid hydrocarbon it will pass through the LQHRM (52) and leave the sensor via the Discharge Screen (55). If the liquid hydrocarbon enters the sensor the LQHRM (52) will absorb it and quickly changes it dielectric properties thus making the presents of liquid hydrocarbon detectable and known. Once the condition of the sensor has been determined electronics within the sensor will transmit that information out of the sensor via the Electrical Connector (54), through electrical interconnections between the network of sensors, to the staff or system tasked with the overall monitoring.

### Other Liquid Hydrocarbon Sensor Applications

A wide variety of other applications are envisaged for the Liquid Hydrocarbon Sensor. Some of these include but are not limited to:

### In Ground Installations

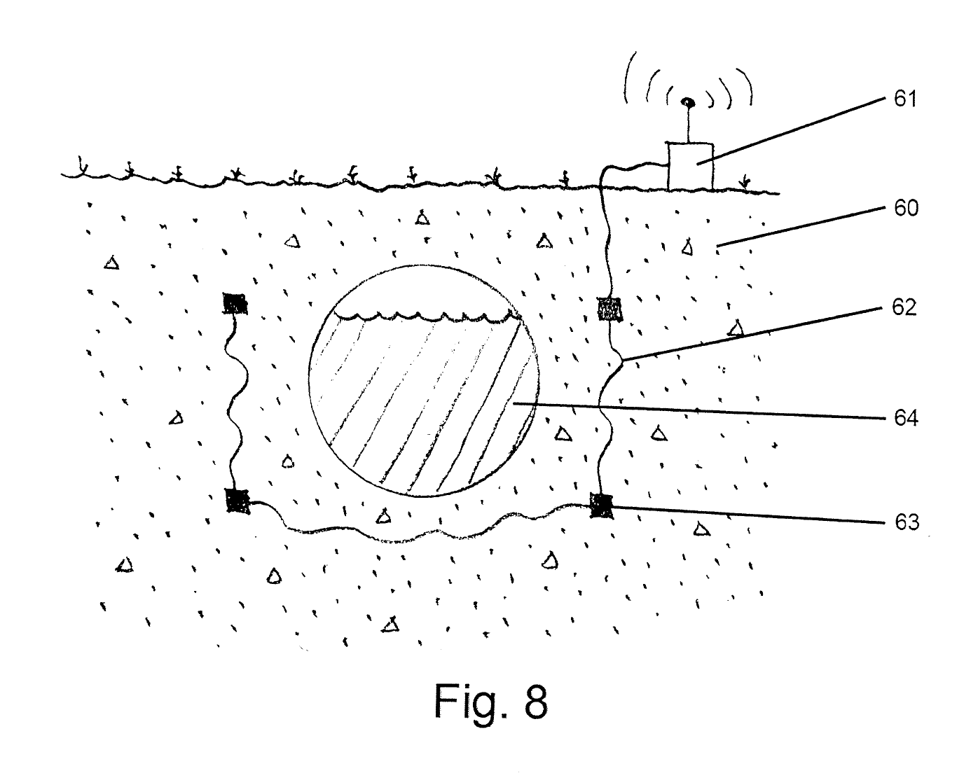

FIG. 8 shows an instance of Burying a network of sensor units (63) in the Ground (60) near and around Buried Tanks (64). Should the tank leak and discharge enough liquid hydrocarbon to allow liquid hydrocarbon to enter any one of the sensor units (63) the presence of liquid hydrocarbon with be communicated through the sensor network's (63) Electrical Interconnections (62) to the Sensor Communications Link (61) and report that an environmentally hazardous event has taken place.

### Ship Bilge

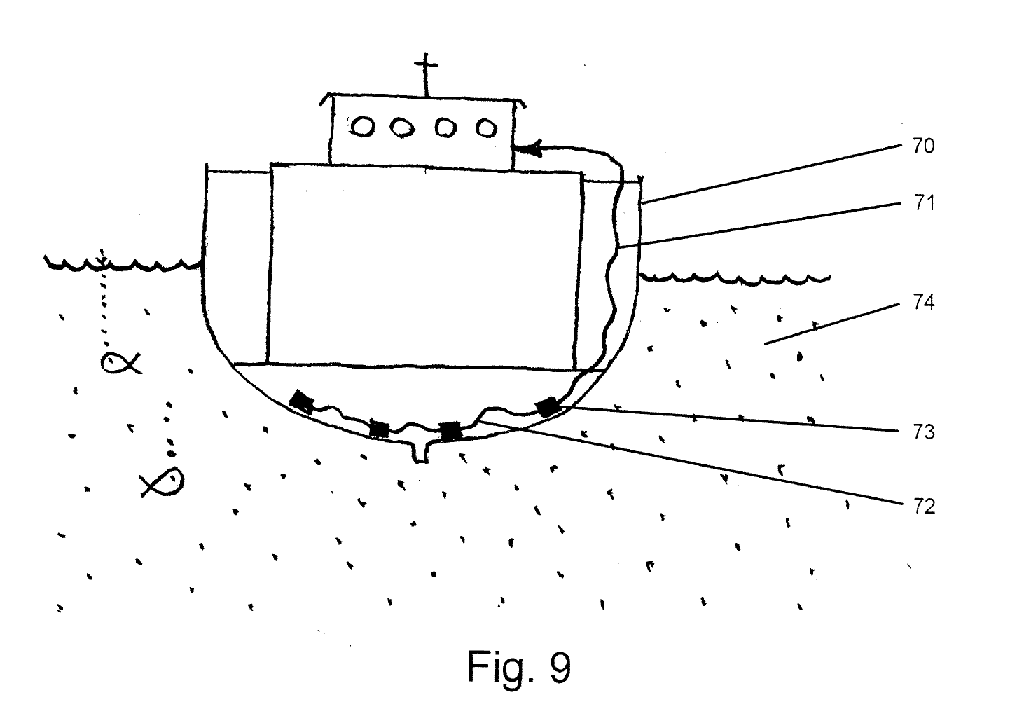

FIG. 9 shows an instance that involves the installation of a network of sensors (73) in strategic locations in the bilges of ships (70). Should liquid hydrocarbon leak into the ship's bulge and enter any one sensor unit (73) a detection signal will be communicated through the sensor network's (73) electrical interconnections (72) to the Sensor Communications Link (71) and ultimately be reported to the ship's bilge monitoring system so that corrective action can be taken before leaks into the surrounding water (74).

### Pipe Covering

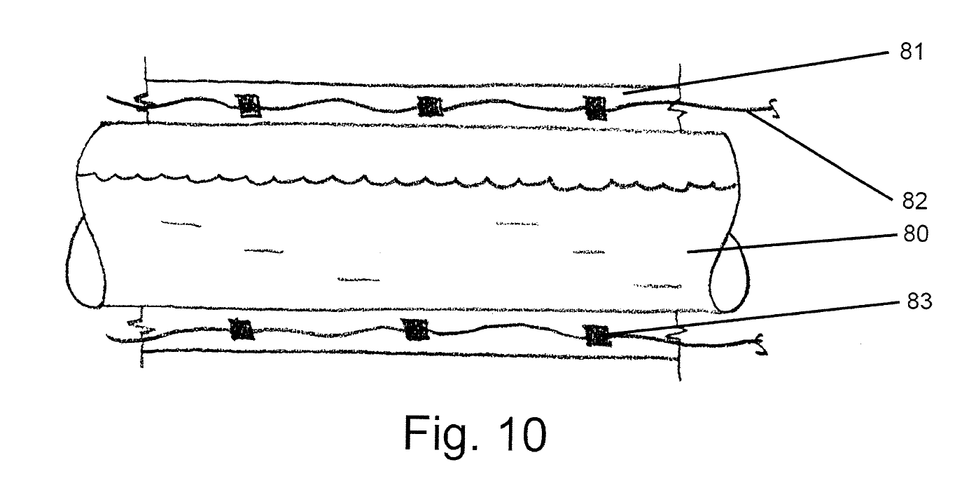

FIG. 10 shows an instance that involves the installation of a network of sensors (83) embedded in the Pipe Covering (81) materials surrounding a pipe (80) carrying liquid hydrocarbon. Should the pipe develop a leak and liquid hydrocarbon enters any one of the sensors (83) a detection signal will be communicated through the sensor network's (83) Electrical Interconnections and Sensor Communications Link (82) to the system monitoring the condition of the pipe. In this way both the fact that a leak has occurred and the approximate location of the leak can be quickly reported and rapid corrective action can take place.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

The scope of the claims should not be limited by the illustrated embodiments set forth as examples, but should be given the broadest interpretation consistent with the description as a whole.

What is claimed is:

1. An apparatus for detecting a selected liquid contaminant, comprising:
a material holder;
a reactive material held by the material holder, the reactive material absorbing a selected liquid contaminant with a measurable change reaction occurring as a result of such absorption; and
a signal generator for generating a signal upon the measurable change reaction exceeding a preset threshold.

2. The apparatus of claim 1, wherein the selected liquid contaminant is a hydrocarbon.

3. The apparatus of claim 1, wherein the signal generator includes a sensor that provides not only an indication that the measurable change reaction has occurred but also an indication of the extent to which the measurable change reaction has occurred.

4. The apparatus of claim 2, wherein the reactive material is hydrophobic and non-reactive to water.

5. The apparatus of claim 2, wherein the reactive material is normally granular allowing passage of liquids and upon reacting with the selected liquid contaminant changes to a gel.

6. The apparatus of claim 2, wherein volumetric expansion occurs during the measurable change reaction of the reactive material.

7. The apparatus of claim 6, wherein a indicator element is provided in close proximity to the reactive material and movement of the indicator element invariably occurs as a result of physical contact during volumetric expansion, the signal generator generating a signal upon movement of the indicator element as a result of volumetric expansion exceeding a preset threshold.

8. The apparatus of claim 7, wherein the indicator element is magnetically responsive and a magnet the interacts with the indicator element and a hall-effect sensor to measure the interaction of the magnet and the indicator element to determine the degree of volumetric expansion which has occurred.

9. The Apparatus of claim 7, wherein the indicator element acts as a switch and makes physical contact to complete an electrical circuit during movement caused by volumetric expansion of the reactive material.

10. The apparatus of claim 2, wherein an optical change occurs during the measurable change reaction of the reactive material.

11. The apparatus of claim 10, wherein the optical change is from opaque to translucent.

12. The apparatus of claim 11, wherein a light source is positioned in a first position relative to the reactive material and a light detector is positioned in a second position relative to the reactive material to detect light passing through the reactive material, the signal generator generating a signal upon a quantity of light being detected by the light detector exceeding a preset threshold.

13. The apparatus of claim 2, wherein a change in dielectric properties occurs during the measurable change reaction of the reactive material.

14. The apparatus of claim 13, wherein there is a change in capacitance.

15. The apparatus of claim 14, wherein there is more than one electrically conductive plate separated by the reactive material and a capacitance detector, the signal generator generating a signal upon the detected capacitance exceeding a preset threshold.

16. The apparatus of claim 1, wherein the material holder is a case with an inlet screen and an outlet screen that allow liquids to pass through the case while screening out debris.

17. The apparatus of claim 1, wherein more than one apparatus is provided, the more than one apparatus being linked by electrical Interconnections the signal generator for generating a signal upon the measurable change reaction in any one of the more than one apparatus exceeding the preset threshold.

18. The apparatus of claim 17, wherein the more than one apparatus are interspersed in a filter substrate to detect filter contamination by the selected liquid contaminant.

19. The apparatus of claim 17, wherein the more than one apparatus are interspersed in ground surrounding a liquid tank to detect leakage from the liquid tank of the selected liquid contaminant.

20. The apparatus of claim 17, wherein the more than one apparatus are interspersed in a bilge of a ship's hull where bilge water collects to detect contamination of the bilge water by the selected liquid contaminant.

21. The apparatus of claim 17, wherein the more than one apparatus are interspersed in a coating surrounding a pipe to detect leakage from the pipe of the selected liquid contaminant.
